(19) United States
(12) Patent Application Publication
(10) Pub. No.: US 2026/0015687 A1
(43) Pub. Date: Jan. 15, 2026

(54) METHOD FOR RECOVERING RARE EARTH METAL

(71) Applicant: IUCF-HYU (INDUSTRY-UNIVERSITY COOPERATION FOUNDATION HANYANG UNIVERSITY), Seoul (KR)

(72) Inventors: Hyun Jung KIM, Seoul (KR); Ilyas SADIA, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 18/992,472

(22) PCT Filed: Jul. 10, 2023
(86) PCT No.: PCT/KR2023/009754

§ 371 Date: Jan. 8, 2025

(30) Foreign Application Priority Data
Jul. 11, 2022 (KR) 10-2022-0085060

(51) Int. Cl. C22B 3/18; C01F 17/20; C12N 1/16; C22B 3/06; C22B 3/38; C22B 3/44; C22B 59/00

(52) U.S. Cl. CPC C22B 3/18; C01F 17/20; C12N 1/16; C22B 3/065; C22B 3/38; C22B 3/44; C22B 59/00; C01P 2002/72

(57) ABSTRACT

The present disclosure describes an eco-friendly bio-based process for effectively recovering a rare earth metal from a rare earth metal source, particularly a low-grade phosphate mineral such as monazite, through solvo-chemical extraction.

### TECHNICAL FIELD

The present disclosure relates to a method of recovering a rare earth metal. More specifically, the present disclosure is directed to an eco-friendly bio-based process for effectively recovering a rare earth metal from a rare earth metal source, particularly a low-grade phosphate mineral such as monazite, through solvo-chemical extraction.

### BACKGROUND ART

Rare earth metals (REMs) are widely used in advanced manufacturing industries such as semiconductors due to their unique physical and chemical properties such as high chemical stability and thermal conductivity, and demand therefor is currently rapidly increasing all over the world. In particular, rare earth metals are mainly used in catalysts, ceramics and glasses, metals and alloys, abrasives, and the like. Among them, cerium (Ce) is known to be the most widely used rare earth metal.

Meanwhile, rare earth metals are not present as elements in nature, but rather as various minerals in which they exist in the form of halides, carbonates, oxides, and phosphates. Currently, approximately 200 minerals are known to contain rare earth elements, but most of them contain small amounts of rare earth metals. Therefore, the number of industrially available minerals is limited.

In this regard, three minerals are known as the main sources of rare earth metal: Bastnaesite, Monazite, and Xenotime. Among them, monazite is a mineral in which cerium (Ce) group rare earth metals occurs in the form of phosphate. It is abundant and found in many areas around the world.

In addition, monazites have chemical stability. Therefore, methods such as concentration (WO 2016-109966 A1), smelting (WO 2009-021389 A1), acid and alkaline decomposition followed by leaching-ion exchange (Korean Patent Publication Laid-open No. 2013-0076261), HCl leaching (U.S. Pat. No. 9,752,213), mechano-chemical leaching (Korean Patent No. 1058567), bio-leaching (Korean Patent No. 1641074), and solvent extraction (Chinese Patent No. 101319275) are known to recover rare earth metals from monazites. Among them, acid or alkaline decomposition is widely used.

Acid decomposition has an advantage of using of inexpensive acids such as sulfuric acid, but may cause equipment corrosion and emits a large amount of corrosive gases, thus resulting in environmental pollution. On the other hand, alkali decomposition is relatively easy to operate and has an advantage of decomposition rate, but is expensive and may cause environmental pollution. Furthermore, acid or alkali decomposition consumes large amounts of energy, and requires large amounts of reagents to neutralize the free acid or alkali left after leaching of the roasted sample.

Alternatives to the alkali decomposition include biological methods using recovery based on microorganisms, such as biosorption, bioleaching, and biomineralization. However, these methods mainly focus on solubilization of phosphate in rare earth minerals using phosphate-solubilizing microorganisms rather than leaching of rare earth metals (Korean Patent No. 1641074).

Thus, there is a need for methods that overcome the limitations of the aforementioned conventional technologies and effectively separate and recover rare earth metals from low-grade resources (or minerals), specifically phosphate minerals such as monazites, in an environmentally friendly manner.

### DISCLOSURE

### Technical Problem

The present disclosure provides an environmentally friendly integrated process that is capable of selectively leaching or extracting rare earths from low-grade rare earth-containing phosphate sources such as monazites using microorganisms, and then of performing effective separation and recovery.

### Technical Solution

In accordance with a first aspect of the present disclosure, there is provided a method of recovering rare earth metals comprising:
a) culturing a phosphate solubilizing microorganism in the presence of a solid rare-earth metal-containing phosphate source, and leaching phosphorus in the solid rare-earth metal-containing phosphate source by metabolic acid discharged from the microorganism to form a phosphorus-containing leachate and a phosphorus-depleted residue, wherein the rare-earth metal-containing phosphate source contains (i) cerium, (ii) at least one rare-earth metal other than cerium, and (iii) iron,
b) culturing a microorganism capable of oxidizing sulfur and iron in the presence of the phosphorus-depleted residue and leaching the iron in the phosphorus-depleted residue by a metabolic lixiviant discharged from the microorganism capable of oxidizing sulfur and iron to form an iron-depleted residue,
c) treating the iron-depleted residue with an acid to leach the rare earth metals and selectively oxidize cerium (III) among the rare earth metals into cerium (IV),
d) extracting the cerium (IV) from the leachate obtained in step c) using an organic solvent to form a cerium-rich extract and a cerium-depleted raffinate, and
e) recovering cerium from the extract.

In an embodiment, the rare earth metal-containing phosphate source may contain 0.05 to 0.6 wt % of phosphorus (P), on an elemental basis.

In an embodiment, the rare earth metal-containing phosphate source may contain 0.5 to 4 wt % of the cerium, 0.1 to 3.5 wt % of the at least one rare earth metal other than cerium, and 5 to 30 wt % of the iron, on an elemental basis.

In an embodiment, the rare earth metal-containing phosphate source may contain up to 25 wt % of an oxide of other metal.

In an embodiment, the rare earth metal-containing phosphate source may be monazite.

In an embodiment, the rare earth metal-containing phosphate source in step a) may have a particle or pulverized material having a size ranging from 50 to 400 mesh.

In an embodiment, the rare earth metal other than cerium may comprise at least one of lanthanum (La) or yttrium (Y).

In an embodiment, the other metal may comprise at least one selected from the group consisting of silicon, titanium, aluminum, zirconium, sodium, potassium, calcium, manganese, and magnesium.

In an embodiment, in step a), the metabolic acid may comprise oxalic acid and the concentration of oxalic acid in the metabolic acid may be at least 200 mM.

In an embodiment, in step a), a part of the rare earth metal in the phosphate source may be precipitated as an organic salt by the metabolic acid and may be contained in the residue.

In an embodiment, culturing in step a) may be performed in the presence of a microorganism initially cultured in a growth medium, wherein a liquid-solid ratio (L/S ratio) of the growth medium/phosphate source may be adjusted in a range of 5 to 15.

In an embodiment, step b) may be performed using a medium supplemented with sulfur and nutrients, wherein a liquid-to-solid ratio of the medium to the phosphorus-depleted residue may be adjusted in a range of 2 to 5.

In an embodiment, in step c), the acid may be nitric acid and ozone-nitration may be involved.

In an embodiment, in step d), the organic solvent may contain an organophosphorus compound immiscible with water.

In an embodiment, a concentration of the organophosphorus compound may be in the range of 0.05 to 0.5 M, and a volume ratio of an organic phase to an aqueous phase (O/A) may be adjusted in a range of 5:1 to 1:5.

In an embodiment, step e) may comprise converting cerium (IV) into cerium (III) using hydrogen peroxide as a reducing agent and then stripping cerium (III) with an acid solution to obtain a cerium-containing acid solution.

In an embodiment, the method may further comprise:
f) recovering rare earth metals other than cerium from the raffinate.

In an embodiment, the method may further comprise precipitating, in the form of cerium oxalate, the cerium contained in the acid solution through the stripping, by use of oxalic acid ions to recover the cerium.

In an embodiment, the rare earth metal other than cerium in the raffinate may comprise at least one of lanthanum (La) or yttrium (Y).

In an embodiment, step f) further may comprise precipitating, in the form of oxalate, rare earth metals other than cerium in the raffinate, by use of oxalic acid ions.

### Advantageous Effects

The process of recovering rare earth metals according to embodiments of the present disclosure is a bio-based process using microorganisms, which can effectively destroy the phosphate structure in a rare earth metal source, for example, low-grade minerals such as monazites to separate and recover rare earth metals without heat treatment, any use of highly concentrated chemicals, or the like, which are involved in the conventional methods, thereby providing an environmentally friendly and sustainable solution. In particular, the process can realize environmental friendliness, low energy intensity, and high efficiency, and is also in line with the ESG strategies of rare earth-related companies, and therefore, extensive commercialization is expected in the future.

### DESCRIPTION OF DRAWINGS

FIG. 1 is a schematic diagram illustrating an example of an integrated process for separating and recovering phosphorus (P), iron (Fe), cerium (Ce), and rare earth metals other than cerium (La, Y), respectively, from a rare earth metal-containing phosphate source.

FIG. 2 is a graph showing a concentration of metabolic acid produced over time and a bioleaching rate during leaching of phosphorus (P) in a rare earth metal-containing phosphate source using microorganisms in Examples.

FIG. 3 is a graph showing an amount of leached iron (Fe) and bioleaching rate over time during leaching of iron in the phosphorus-depleted residue after separation of phosphorus (P) from the rare earth metal-containing phosphate source using microorganisms.

FIG. 4 is a graph showing a leaching rate of each of three rare earth metals (Ce, La, and Y) depending on the concentration of nitric acid as a result of ozone-nitration of iron-depleted residue after separation of iron (Fe).

FIG. 5 is a graph showing an extraction rate of three rare earth metals (Ce, La, and Y) in the leaching solution depending on the concentration of organic extractant.

FIG. 6 is a graph showing a cerium stripping rate depending on the concentration of hydrochloric acid during stripping of a cerium (IV)-containing extract using hydrogen peroxide (concentration: 0.1 mol/L).

FIG. 7 shows an XRD pattern of a cerium oxalate precipitate formed by reaction with oxalate ions after stripping.

FIG. 8 is a graph showing a precipitation rate of rare earth metals depending on the concentration of oxalate ions during the process of forming precipitates (oxalate precipitates) of rare earth metals (La, Y) other than cerium by adding ammonium oxalate to the raffinate formed by cerium extraction.

FIG. 9 shows an XRD pattern of precipitates of mixtures of oxalate and rare earth metals (La, Y).

### BEST MODE

The present disclosure can be achieved by the following description. It should be understood that the following description describes preferred specific examples of the present disclosure and the present disclosure is not necessarily limited thereto. In addition, the attached drawings are merely provided for better understanding, the present disclosure is not limited thereto and details regarding each component may be appropriately understood by the specific goal of the related description described below.

The terms used herein are defined as follows.

The term “rare earth metal” is a general term for 17 elements including scandium and yttrium in Group 3A of the periodic table, and 15 lanthanide elements with atomic numbers from 57 to 71, which have similar chemical properties and generally have an oxidation number of +3. In addition, rare earth metals may be generally classified into light rare earth metals (elements) and heavy rare earth metals (elements), where the four elements from lanthanum (La) having an atomic number of 57 to neodymium (Nd) having an atomic number 60 belong to light rare earth elements, while the remaining 13 elements belong to heavy rare earth elements.

The term “bioleaching” may generally refer to a process of inducing dissolution of metals from a mineral source using microorganisms, specifically a process of converting a solid metal into its water-soluble form.

The term “culture medium” may refer to an aqueous solution of nutrients available for cell growth.

The term “phosphate solubilizing microorganism (PSM)” may refer to a microorganism that converts immobilized phosphorus into soluble form.

The term “extraction” may refer to transfer of a specific component, specifically a metal, from one phase to another.

The term “stripping” may refer to a process of separating or removing a particular metal from a liquid medium or solvent, and “selective stripping” may refer to a process of separating or removing a particular metal from a liquid medium or solvent containing multiple metals.

It should be understood that, when a numerical range is specified herein as a lower limit and/or an upper limit, any sub-combination within that numerical range is also disclosed. For example, the expression “1 to 5” may include 1, 2, 3, 4, and 5, as well as any sub-combination therebetween.

It should be understood that, when one element is referred to as being “connected” to another element, the elements may be directly connected to each other or may be connected to each other via the other element unless otherwise defined.

Similarly, it should be understood that, when one element is referred to as “contacting” another element, the elements may directly contact each other or may contact each other via the other element.

It will be further understood that the term “comprise” when used herein specifies the presence of stated element, but does not preclude the presence or addition of one or more other elements.

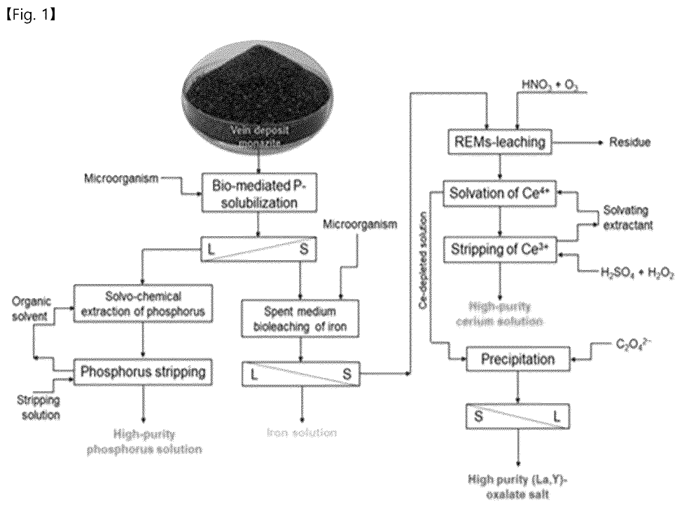

According to one embodiment of the present disclosure, a process of bioleaching and solvo-chemical extraction using microorganisms is provided to separate and recover rare earth metals from a rare earth metal-containing phosphate source. An exemplary process for separating and recovering phosphorus (P), iron (Fe), cerium (Ce), and rare earth metals other than cerium (La, Y) therefrom is shown in FIG. 1.

Referring to the drawing, a solid rare earth metal source is provided as a starting material. In the illustrated embodiment, the solid rare earth metal source may be a phosphate source that contains cerium and rare earth metals other than cerium, and further contains iron, and more typically, a naturally derived low-grade mineral. For example, the rare earth metal source may be a mineral, specifically, monazite. Monazite may be a phosphate mineral generally represented by formulas such as (Ce,La,Th)PO₄ or (Ce, La, Nd, Th)PO₄.

The content of phosphorus (P) in the rare earth metal source may be, for example, about 0.05 to 0.6 wt %, specifically about 0.07 to 0.3 wt %, more specifically about 0.09 to 0.2 wt %, and particularly specifically about 1 wt %, on an elemental basis.

Meanwhile, the content of cerium (on an elemental basis) in the rare earth metal salt source may be, for example, about 0.5 to about 4 wt %, specifically about 0.5 to about 3 wt %, more specifically about 0.7 to about 2 wt %, and particularly specifically about 1 wt %. In addition, examples of rare earth metals other than cerium include lanthanum (La) and yttrium (Y), and the rare earth metal salt source may contain at least one of lanthanum (La) or yttrium (Y). However, the present embodiment is not limited thereto and the rare earth metal salt source may contain other rare earth metals (e.g., Pr, etc.) instead of or in addition to lanthanum and/or yttrium. For example, the content (on an elemental basis) of rare earth metals other than cerium may be, for example, in the range of about 0.1 to 3.5 wt %, specifically about 0.3 to 2 wt %, more specifically about 0.5 to 1 wt %. In addition, the content (on an elemental basis) of iron in the rare earth metal source may be, for example, in the range of about 5 to 30 wt %, specifically about 8 to 20 wt %, more specifically about 10 to 15 wt %.

According to an exemplary embodiment, when the rare earth metal source is in the form of a mineral, it may contain other metals, in addition to the aforementioned phosphorus, rare earth metals and iron, and the other metals are not particularly limited and may, for example, include at least one selected from silicon, titanium, aluminum, zirconium, sodium, potassium, calcium, manganese, magnesium, and the like. The content of other metals may be, for example, up to about 25 wt %, specifically about 10 to 20 wt %, and more specifically around 20 wt %, based on the oxide.

The content of each component in the rare earth metal source is provided as an example and may vary depending on the origin region, or the like.

In this regard, an exemplary composition of monazite available from a specific origin as a rare earth metal source is shown in Table 1 below (wt %).

TABLE 1

| Fe₂O₃ | Al₂O₃ | SiO₂ | TiO₂ | Y₂O₃ | La₂O₃ | CeO₂ | PO₄ | Na₂O | MgO | K₂O | MnO | CaO | ZrO₂ |
|---|---|---|---|---|---|---|---|---|---|---|---|---|---|
| 28.4 | 8.1 | 34.7 | 11.3 | 0.13 | 0.68 | 1.06 | 0.86 | 0.8 | 1.6 | 1.3 | 0.6 | 5.1 | 5.7 |

According to an exemplary embodiment, the solid rare earth metal-containing phosphate source may have a pulverized material (form) or particle form so that subsequent leaching (bioleaching) can be performed effectively. In this case, the size of the pulverized material or particle may be determined depending on sieving, and may be, for example, in the range of about 50 to 400 mesh, specifically about 70 to about 350 mesh, and more specifically about 80 to about 325 mesh.

Meanwhile, a pulverization means known in the art may be used for minerals such as monazite. For example, the pulverization means may be a roller-type pulverizer, a vibrating mill, a ball mill, a pot mill, a hammer mill, a pulverizer, a gyratory mill, or the like, but is not limited thereto.

### Bioleaching of Phosphorus (P)

Referring to FIG. 1, the phosphate source is subjected to bioleaching in which phosphorus is leached using phosphate solubilizing microorganisms.

As shown in FIG. 1, the reason why phosphorus (P) is preferentially leached and separated from rare-earth metal-containing phosphate sources, especially monazite, before other ingredients or elements is as follows: in monazite, phosphate acts as a host matrix for rare-earth metals, and the rare-earth metals remain in the center with a distorted coordination sphere surrounded by eight oxides of phosphate. Therefore, in order to leach rare-earth metals with a lixiviant, first, the phosphate structure may be advantageously destroyed.

In the illustrated embodiment, the phosphate solubilizing microorganism (PSM) may be selected from those capable of leaching rare-earth metals by converting phosphorus in the phosphate source into a solubilized form, through the action of metabolic acids, which act as lixiviants, produced during culturing in the presence of the phosphate source. For example, the phosphate solubilizing microorganism may include at least one selected from the group consisting of the genus Aspergillus and the genus Penicillium. In addition, various Penicillium species, heterotrophic bacterial species, and the like may be used. Examples thereof include Pseudomonas rhizosphaerae, Pseudomonas putida, Pseudomonas fluorescens, Bacillus megaterium, Paenibacillus polymyxa, Ensifer meliloti, Azospirillum brasilense, Mesorhizobium ciceri, Acetobacter aceti, Pseudomonas aeruginosa, and the like. and these Penicillium species may be used alone or in combination. However, the types of Penicillium species listed above are provided as examples.

Phosphorus (P) is an essential element for living organisms, acts to maintain major cellular reactions, carbon and amino acid metabolic processes, and is essential for ATP, enzymatic reactions, and energy transfer. In this case, the phosphate solubilizing microorganisms may discharge an insoluble phosphorus in the phosphate source into its soluble form by acidification, chelation, exchange reaction, or the like.

According to an exemplary embodiment, in order to culture the phosphate solubilizing microorganisms, the medium may be a growth medium, specifically, a modified growth medium, and the phosphate solubilizing microorganisms may be treated with the phosphate source such that the microorganisms initially cultured in a growth medium are combined with the phosphate source and then are grown and cultured. For example, the culture of the microorganisms may be performed in a fed-batch culture manner and the fed-batch culture may refer to a process in which at least one nutrient (e.g., sucrose) is intermittently or continuously added to the reactor during the culture process. For example, the concentration of microbial spores in the initial growth medium may be, for example, at least about 3×10⁶ spores/mL, specifically about 3×10⁸ to about 3×10¹⁰ spores/mL, more specifically about 1×10⁹ to about 5×10⁹ spores/mL, and particularly specifically about 3×10⁹ spores/mL, but this may be understood as an example. In this case, the phosphorus ingredient in the phosphate source acts as a source that provides phosphorus necessary for culturing, and thus may eliminate the necessity of a separate supply of phosphorus. The ingredients of the medium used for microbial growth in this embodiment are known in the art and thus a separate detailed description will be omitted. Meanwhile, in an exemplary embodiment, the liquid/solid ratio (L/S ratio) of the growth medium/phosphate source (solid phase) may be adjusted in consideration of the amounts of the medium and the phosphate source, for example, about 5 to about 15, specifically about 5 to about 13, more specifically about 8 to about 12, and particularly specifically about 10.

As the microbial culture time increases, the amount of metabolic acid produced increases, causing phosphorus to be leached from the phosphate source. An exemplary reaction accompanying this leaching process is depicted in the following Reaction Scheme 1.

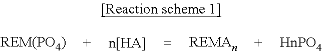

According to an exemplary embodiment, the metabolic acid produced by the microorganism may comprise, for example, oxalic acid (C₂H₂O₄) and may further comprise at least one selected from gluconic acid, citric acid, formic acid, butyric acid, maleic acid, and the like. In this regard, the concentration of the metabolic acid produced by the microorganism may be, for example, at least about 200 mM, specifically about 230 to 1200 mM, more specifically about 500 to 900 mM, and particularly specifically about 840 mM, and at this time, the proportion of oxalic acid may be, for example, at least about 35%, specifically about 40 to 80%, more specifically about 50 to 70%, and particularly specifically about 56%, based on a total mole of the metabolic acid. For example, the concentration of oxalic acid in the metabolic acids produced by microbial culture may be, for example, at least about 200 mM, specifically about 300 to 900 mM, more specifically about 350 to 600 mM, and particularly specifically about 470 mM.

In an exemplary embodiment, the bioleaching conditions of the phosphorus ingredient are not particularly limited because they may vary depending on the composition, properties, and the like of the starting material, i.e., the phosphate source. As an example, the temperature may be controlled, for example, at about 20 to 45 (±1) ° C., specifically about 25 to 40 (±1) ° C., more specifically about 30 to 35 (±1) ° C., and particularly specifically about 30 (±1) ° C. Further, the pH may be controlled, for example, in a range of about 4 to about 7, specifically about 4.5 to about 6.5, and more specifically about 5 to about 6. In addition, the leaching of the phosphorus ingredient may be performed under stirred or non-stirred conditions and may be preferably performed under stirred conditions. Moreover, the leaching time may be determined within a range of, for example, about 2 to about 20 days, specifically about 3 to about 14 days, more specifically about 3 to about 12 days, and particularly specifically about 8 to about 10 days, but is not limited thereto.

In an exemplary embodiment, through the bioleaching process using the aforementioned phosphate solubilizing microorganism, at least about 70%, specifically at least about 75%, more specifically about 80 to 90%, and particularly specifically about 83% of the total phosphorus (P) element in the phosphate source may be solubilized.

In addition, in the bioleaching process of the phosphorus ingredient by metabolic acid, not only phosphorus (P), but also one or two other ingredients in the rare earth metal-containing phosphate source may be at least partially leached or dissoluted. The ingredients further leached in this way may be alkali metals (e.g., sodium, potassium, etc.), alkaline earth metals (e.g., calcium, etc.), rare earth metals, iron, and the like. However, the amount of each ingredient leached (or dissoluted) along with phosphorus (P) may vary depending on the composition of the phosphate source, or the like, and is therefore not limited to a specific range.

In addition, the rare earth metal leached along with phosphorus may be combined with a metabolic acid to form a complex and may be converted from an inorganic compound (oxide) to an organic compound (e.g., rare earth oxalate), and this organic substance or organic salt may be precipitated and incorporated into the phosphorus-depleted residue, or may be provided along with the phosphorus-depleted residue for the subsequent treatment process. An example of the precipitation of rare earth metals (e.g., conversion to oxalate) may be depicted by the following Reaction Schemes 2 and 3.

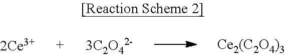

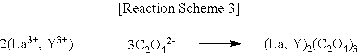

Meanwhile, according to an exemplary embodiment, phosphorus in the leachate may be obtained in the form of a solution containing high purity phosphorus through an additional separation and purification process. As a method of separating and purifying phosphorus, a solvo-chemical method, specifically, extraction and stripping using an organic solvent as illustrated, may be used. In this case, as an extractant, at least one organic solvent selected from, for example, an oxide phosphine compound having a long carbon skeleton (about 16 to 48 carbon atoms, specifically about 25 to 35 carbon atoms) may be used and a phosphorus-rich extract is generated by extraction. Then, at least one stripping solution selected from, for example, inorganic acids such as hydrochloric acid, nitric acid, sulfuric acid, and perchloric acid is added to the phosphorus-rich extract, so that a solution containing high purity phosphorus may be obtained. In addition, the organic solvent in the extract may be recycled and reused as an extractant in the extraction before stripping.

According to an exemplary embodiment, the extraction conditions are not particularly limited and the volume ratio of the organic phase to the aqueous phase may be adjusted in a range of about 1:5 to about 5:1, specifically about 1:3 to about 3:1, more specifically about 1:2 to about 2:1. In addition, the concentration of the extractant (based on the organic phase) may be, for example, about 0.1 to about 0.5 M, specifically about 0.15 to about 0.4 M, more specifically about 0.2 to about 0.3 M, and particularly specifically about 0.25 M. Meanwhile, the equilibration time may be, for example, about 1 to 30 minutes, specifically about 2 to 20 minutes, more specifically about 4 to about 10 minutes, and particularly specifically about 5 minutes. The extraction temperature may be, for example, about 20 to 40° C., specifically about 22 to about 35° C., more specifically about 24 to about 30° C., and particularly specifically about 25° C. In addition, the phase separation time may be, for example, determined to about 5 to about 30 minutes, specifically about 7 to about 25 minutes, more specifically about 9 to about 15 minutes, and particularly specifically about 10 minutes.

According to an exemplary embodiment, although not particularly limited, the volume ratio of the organic phase to the aqueous phase may be controlled in a range of about 1:5 to about 5:1, specifically about 1:3 to about 3:1, and more specifically about 1:2 to about 2:1. In addition, the acid concentration may be, for example, about 1 to about 3 M, specifically about 1.4 to about 2.7 M, more specifically about 1.8 to about 2.5 M, and particularly specifically about 2 M. In addition, hydrogen peroxide may be used during stripping, and at this time, the concentration of hydrogen peroxide may be, for example, about 0.05 to 0.2 M, specifically about 0.08 to 0.15 M, more specifically about 0.1 M. The contact time may be, for example, about 1 to about 20 minutes, specifically about 3 to about 10 minutes, more specifically about 4 to about 6 minutes, and particularly specifically about 5 minutes. Meanwhile, the stripping temperature may be, for example, about 20 to 40° C., specifically about 22 to 35° C., more specifically about 24 to 30° C., and particularly specifically around 25° C. In addition, the phase separation time may be determined to, for example, about 5 to about 30 minutes, specifically about 7 to about 25 minutes, more specifically about 9 to about 15 minutes, and particularly specifically about 10 minutes. [96]

### Bioleaching of Iron (Fe)

Referring to FIG. 1 again, bioleaching using microorganisms may be performed to separate and recover iron from the phosphorus-depleted residue (specifically, iron-rich residue) left after phosphorus leaching.

According to an exemplary embodiment, microorganisms usable for iron leaching are microorganisms capable of oxidizing sulfur and iron and release metabolically produced lixiviants. Such microorganisms may include at least one selected from the group consisting of Alicyclobacillus genus and Sulfobacillus genus. Specifically, the genus Alicyclobacillus is a microorganism belonging to the Alicyclobacillaceae family, which includes Gram-positive or Gram-variable microorganisms, and examples thereof include A. acidiphilus, A. disulfidooxidans, A. montanus, A. sacchari, and the like. In addition, examples of microorganisms belonging to the genus Sulfobacillus include Sulfobacillus acidophilus, Sulfobacillus benefaciens, Sulfobacillus disulfidooxidans, Sulfobacillus sibiricus, Sulfobacillus thermosulfidooxidans, and Sulfobacillus thermotolerans.

In the illustrated embodiment, the lixiviant formed as a metabolite during the microbial culture process may be sulfuric acid produced by oxidation of the elemental sulfur or sulfur in the presence of oxygen by the microorganism, as depicted in the following Reaction Scheme 4. In this case, oxygen may be provided by external aeration.

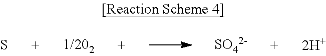

According to an exemplary embodiment, microorganisms for leaching iron may be cultured while supplementing or supplying sulfur and nutrients (e.g., sucrose) to the spent medium left after the phosphorus leaching. At this time, the mechanism for leaching (solubilizing) iron from the phosphorus-depleted residue may be depicted in the following Reaction Scheme 5.

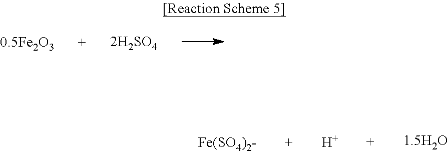

For example, sulfur may be obtained from various sources, and may, for example, be biological sulfur collected from a sedimentation tank of a wastewater treatment plant. In addition, as described above, organic acid ions (e.g., oxalate ions) that may be precipitated in the form of organic salts (e.g., oxalate) during the leaching process and may exist in the residue may act as catalysts for solubilization of iron.

According to an exemplary embodiment, the liquid-to-solid ratio of the medium/residue (phosphorus-depleted residue) during iron leaching may be adjusted in consideration of the amounts of the medium and residue, for example, in the range of about 2 to 5, specifically about 2.3 to 4, more specifically about 2.5. In addition, the leaching conditions of iron in the phosphorus-depleted residue may vary depending on various factors (the content and the properties of iron in the phosphate source as a starting material or the like) and thus are not particularly limited, but the temperature may be controlled, for example, in a range of about 30 to 60° C., more specifically, in a range of about 40 to 55° C., and specifically, in a range of about 50° C., and the pH may be controlled, for example, in a range of about 1.1 to about 2.8, specifically, in a range of about 1.3 to about 2, and more specifically, in a range of about 1.5. Furthermore, the leaching of iron may be performed under stirred or non-stirred conditions and preferably under stirred conditions. In addition, the leaching time may be determined, for example, in a range of about 1 to about 21 days, specifically, in a range of about 2 to about 10 days, and more specifically, in a range of about 4 to about 8 days, but is not limited thereto.

In an exemplary embodiment, during iron bioleaching, the amount of iron released increases over time. At least about 50%, specifically at least about 90%, more specifically at least about 99% of the elemental iron in the phosphorus-depleted residue may be solubilized, and leached or dissoluted to form an iron-containing solution in a liquid phase and a solid iron-depleted residue.

In addition, other ingredients that are not completely removed during phosphorus leaching, such as alkali metals (e.g., sodium or potassium) and alkaline earth metals (e.g., calcium) may also be leached and removed. However, a proportion of the leaching of rare earth metals may be limited, for example, to less than about 2%, specifically less than about 1.5%, more specifically less than about 1%.

The iron-containing solution prepared by leaching of the phosphorus-depleted residue may be separated, and then subjected to further treatment, e.g., precipitation, solvent extraction, ion exchange, or the like, to recover iron. Such separation and recovery methods are well known in the art and thus detailed descriptions are omitted.

### Separation and Recovery of Cerium

Referring to FIG. 1, cerium is selectively separated and recovered among the rare earth metals in the iron-depleted residue.

Specifically, the iron-depleted residue is treated with an acid (aqueous acid solution) to leach rare earth metals, and cerium (III) may be selectively oxidized among the leached rare earth metals and converted to cerium (IV).

According to an exemplary embodiment, nitric acid may be used as an acid for leaching rare earth metals. At this time, the concentration of the acid may be determined considering the content of cerium, and may be adjusted in the range of, for example, about 0.2 to 8 M, specifically about 0.5 to 6 M, more specifically about 1 to 5 M, more specifically about 2 to 4.5 M, and particularly specifically about 3 to 4 M. Typically, the leaching of rare earth metals tends to increase as the concentration of the acid increases.

Meanwhile, along with the leaching of rare earth metals in the iron-depleted residue, cerium having an oxidation number of +3, i.e., cerium (III), among rare earth metals, may be selectively oxidized to cerium (IV).

As an oxidizing agent to oxidize the leached cerium (III) to cerium (IV), ozone, peroxydisulfate, or oxygen may be used alone or in combination, and the oxidizing agent may specifically be ozone. According to a particular embodiment, ozone nitration using nitric acid (nitric acid solution) and ozone may be performed. In this case, the oxidation may be performed in accordance with the following Reaction Scheme 6.

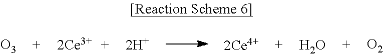

In the oxidation reaction described above, cerium is selectively oxidized, while the remaining rare earth metals (e.g., lanthanum and/or yttrium) remain with an oxidation number of +3. At this time, a catalyst for converting ozone into molecular oxygen may be used. For example, a catalyst bed including CuO and MnO₂ may be used.

According to an exemplary embodiment, the liquid-to-solid ratio of the acid/iron-depleted residue may be adjusted considering the amount of acid and residue, for example, in a range of about 2 to 20, specifically about 5 to 15, more specifically about 8 to 12, and particularly specifically about 10.

In addition, the conditions for leaching of rare earth metals and selective oxidation of cerium are not particularly limited, and the temperature may be adjusted, for example, in a range of about 25 to about 75 (±1) ° C., specifically about 30 to 70 (±1) ° C., more specifically about 45 to about 60 (±1) ° C., and particularly specifically about 55 (±1) ° C. In addition, the treatment time may be adjusted in the range of, for example, about 1 to 24 hours, specifically about 2 to 15 hours, more specifically about 3 to about 10 hours, and particularly specifically about 4 to 6 hours, and this may be provided as an example.

According to an alternative embodiment, instead of ozone nitration, selective oxidation of cerium may be used, where sulfuric acid (H₂SO₄) leaching may be carried out using sulfuric acid roasting. However, this alternative treatment requires a high concentration of acid. Therefore, the ozone-nitration is preferred.

As described above, when the rare earth metal is leached using an acid, and cerium among the rare earth metals in the leachate is selectively oxidized, at least about 60%, specifically at least about 80%, more specifically at least about 90% of the cerium element in the iron-depleted residue may be leached into the acid (acid solution). In addition, at least about 80%, specifically at least about 85%, more specifically at least about 90%, especially specifically at least about 90.1% of the leached cerium having an oxidation number of +3 (cerium (III)) may be oxidized into cerium having an oxidation number of +4 (cerium (IV)). In addition, at least about 80%, specifically at least about 90%, more specifically at least about 98% of other rare earth elements (e.g., lanthanum and/or yttrium) in the iron-depleted residue may be leached.

### Separation and Recovery of Cerium

Referring to FIG. 1, cerium (IV) in a rare earth metal-containing acid leachate is separated (extracted) and recovered.

In the illustrated embodiment, cerium (IV) may be separated from other rare earth metals having an oxidation number of +3 using an organic solvent, for example, an organophosphorus compound immiscible with water, as an extractant in the acid leachate. Representative examples of such an organophosphorus compound includes tri-alkyl phosphine oxide-based compounds, of which at least one may be used. For example, the organophosphorus extractant is commercially available under the trade name Cyanex 923. Cyanex 923 is a mixture of four organic phosphine oxides, and contains dioctyl-monohexyl phosphine oxide (R′R2P═O; 31 wt %), mono-octyl dihexyl phosphine oxide (R′2RP═O; 42 wt %), tri-hexyl phosphine oxide (R′3P═O; 14 wt %), and tri-n-octyl phosphine oxide (R3P═O; 8 wt %).

Meanwhile, the organophosphorus compound may be added or dissolved in a solvent, such as an aromatic solvent, for extraction. Such an aromatic solvent is petroleum hydrocarbon having 9 to 11 carbon atoms, specifically, about 10 carbon atoms, and the petroleum hydrocarbon may be used alone or in combination. In addition, a phase modifier may be used in combination with the organophosphorus compound, and the phase modifier may include at least one selected from, for example, n-heptane, n-decanol, and the like and may be used in an amount of, for example, about 1 to 10 vol %, specifically about 3 to 8 vol %, more specifically about 4 to 6 vol %, based on the organic phase. The composition of the extractant described above may be provided as an example.

According to an exemplary embodiment, the concentration of the organophosphorus compound (based on the organic phase) during extraction may be adjusted in a range of, for example, about 0.05 to about 0.5 M, specifically about 0.15 to about 0.4 M, more specifically about 0.2 to about 0.3 M, but is not limited thereto.

According to an exemplary embodiment, the volume ratio of the organic phase/aqueous phase (O/A) in the extraction step may be, for example, in a range of about 5:1 to about 1:5, specifically about 4:1 to about 1:4, more specifically about 3:1 to about 1:3, and particularly specifically about 2:1. Considering the composition of the organophosphorus compound and the distribution of elements in the aqueous phase, the volume ratio may be advantageously adjusted within the range described above. In addition, the extraction temperature is not particularly limited and may be, for example, in a range of about 10 to about 40° C., specifically about 20 to about 30° C., and more specifically room temperature.

As such, through the extraction treatment of the rare earth metal-containing leachate, at least about 95%, specifically at least about 98%, more specifically at least about 99% of the cerium (IV) element in the leachate is extracted, while about 5% or less, specifically about 4% or less, more specifically about 3.5% or less of other rare earth metals having an oxidation number of +3 is extracted. Accordingly, most of cerium (IV) is contained in the extract, while most of the remaining rare earth metal (specifically having an oxidation number of +3) remains in the raffinate.

According to the illustrated embodiment, cerium with high purity is recovered from the cerium-rich extract obtained by separating cerium (IV) through extraction as described above.

Typically, the recovery of cerium may be performed by converting (reducing) cerium (IV) back to cerium (III) and stripping cerium (III) using an acid. At this time, the acid may include at least one selected from inorganic acids, such as nitric acid, hydrochloric acid, sulfuric acid, and perchloric acid. For example, the concentration of the acid may be determined considering the concentration of cerium (III), for example, in the range of about 0.2 to 3 M, specifically about 0.5 to 2.5 M, more specifically about 1 to 2 M, but this is provided as an example. However, as the concentration of the acid increases, the stripping of cerium may increase.

Meanwhile, hydrogen peroxide may be used as a reducing agent in reductive stripping for stripping cerium (IV) or Ce⁴⁺ from an organic phase. Hydrogen peroxide may usually act as a strong oxidizing agent and a strong reducing agent, and its standard potential is shown in the following Reaction Schemes 7 to 9.

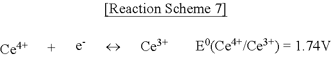

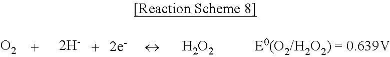

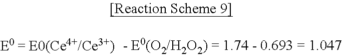

As can be seen from Reaction Scheme, Ce⁴⁺ may be reduced to Ce³⁺ by hydrogen peroxide, which is advantageous in that no other metal impurities are incorporated during the reduction process. The Ce³⁺ formed by the reducing agent in this way may be stripped into an aqueous phase using an acid solution. According to an exemplary embodiment, the concentration of hydrogen peroxide may be determined in a range of, for example, about 0.01 to about 1 M, specifically about 0.05 to 0.5 M, more specifically about 0.08 to about 0.2 M, and particularly specifically around about 0.1 M.

Through the reductive stripping, at least about 60%, specifically at least about 90%, more specifically at least about 95% of the cerium element in the extract may be stripped. As a result, an aqueous cerium solution may be formed. In order to recover cerium from the obtained cerium acid solution (aqueous solution), for example, an organic acid ion, specifically an oxalic acid ion (or oxalate ion), may be supplied to and reacted with an aqueous cerium solution to form a precipitate of an organic acid salt of cerium (specifically, oxalate), specifically, a high-purity cerium salt. For example, a source of the oxalic acid ion may, for example, include at least one of ammonium oxalate, oxalic acid, sodium oxalate or potassium oxalate. In this case, the concentration of oxalate ions may be determined depending on the amount of cerium in the cerium aqueous solution and may, for example, be in the range of about 0.002 to about 2 M, specifically about 0.005 to about 1 M, more specifically about 0.01 to about 0.05 M, and particularly specifically about 0.02 to about 0.03 M. However, the above numerical range may be provided as an example.

In addition, the temperature condition during the formation of the precipitate is not particularly limited and may be controlled in the range of about 20 to about 90° C., specifically about 50 to about 85° C., and more specifically about 60 to about 80° C.

Meanwhile, the organic phase, i.e., the organic solvent, left after stripping of cerium may be recycled and used as an extractant for extraction of cerium (IV) in the previous step.

Separation and Recovery of Rare Earth Metals Other than Cerium

Referring to FIG. 1 again, the raffinate separated from the extract in the process of extracting cerium is in a cerium-depleted state and mainly contains rare earth metals other than cerium (specifically, rare earth metals having an oxidation number of +3). According to an exemplary embodiment, the method may further comprise recovering rare earth metals other than cerium from the cerium-depleted raffinate.

In this case, similar to the procedure for recovering cerium, to recover rare earth metals other than cerium contained in the raffinate, for example, an organic acid ion, specifically an oxalic acid ion (or oxalate ion) is supplied and reacted to form a precipitate of an organic acid salt (specifically, oxalate) of a rare earth metal (e.g., lanthanum and/or yttrium). As a result, high-purity rare earth metal may be recovered.

According to an exemplary embodiment, when oxalate of a rare earth metal other than cerium is formed, the concentration of oxalate ion may be, for example, in a range of about 0.01 to 0.1 M, specifically about 0.02 to about 0.07 M, more specifically about 0.03 to about 0.05 M. However, the numerical range may be provided as an example. In addition, the precipitate formation may be performed under elevated temperature conditions, for example, at a temperature controlled in a range of about 60 to about 95° C., specifically about 65 to 90° C., more specifically about 70 to about 85° C.

As such, through the precipitation process, at least about 50%, specifically at least about 80%, more specifically at least about 95%, and even about 98% of the rare earth elements (having an oxidation number of +3) such as lanthanum and/or yttrium in the raffinate may be obtained as a precipitate (or coprecipitate). At this time, the obtained rare earth metal may be used as a precursor for the production of a fluorescent substance or as a catalyst precursor.

The present disclosure will be more clearly understood by the following examples and the following examples are merely provided for illustration and should not be construed as limiting the scope of the disclosure.

### MODE FOR INVENTION

### Example

### A. Materials

The materials used in this example are shown in Table 2 below.

TABLE 2

| Material name | Manufacturer |
|---|---|
| Glucose | ACS reagent chemicals |
| NaNO₃ | Sigma-Aldrich |
| MgSO₄•7H₂O | Sigma-Aldrich |
| KCl | Sigma-Aldrich |
| (NH₄)₂SO₄ | Sigma-Aldrich |
| K₂HPO₄ | Sigma-Aldrich |
| Yeast extract | Sigma-Aldrich |
| NaOH | Junsei Chemical Co. Ltd. |
| H₂SO₄ | Daejung |
| HCl | Daejung |
| HNO₃ | Riedel-deHaen |
| Organophosphorus | Cyantec. Inc. Canada, |
| compounds | Solvay |
| TBP | Sigma-Aldrich |
| Isododecanol | Shanghai Chemicals |
| C10 | Shanghai Chemicals |
| H₂O₂ | Merck |
| C₂H₈N₂O₄ | Sigma Aldrich |

### B. Analysis Method

The analysis in this example was performed as follows.

XRD pattern was analyzed using X'pert PRO PANalytical.

The content of metal elements in the aqueous phase or organic phase was analyzed by inductively coupled plasma-induced emission spectroscopy using iCAP 7400 Duo (Thermo Scientific, USA).

### Example 1

The vein-deposit monazite ore having the composition shown in Table 1 was pulverized to a size of 80 to 325 mesh using a ball mill and phosphorus was bioleached as shown in FIG. 1. The phosphorous solubilizing microorganism used herein was a mixture of A. japonicus, A. aculeatus, A. niger, P. cinnamopurpureum, and P. chrysogenum.

100 g of a monazite ore sample was placed in a 1.5 L bioreactor at a constant stirring speed of 200 rpm and an operating temperature of 3° C. to perform bioleaching. At this time, the initially cultured microorganisms (3×10⁹ spores/mL) were cultured in a modified growth medium, and the medium contained 9 to 55 g/L of sucrose, 6.0 g/L of NaNO₃, 0.52 g/L of MgSO₄·7H₂O, 0.52 g/L of KCl and 1.6 g/L of an yeast extract.

A monazite ore, which is the sole source of phosphate, was added at a liquid-solid ratio (medium:ore) of 10. Initially, the pH of the medium was adjusted to 4.5 (using 5 wt % dil. H₂SO₄). After culture for 48 hours, the pH was adjusted again to 6.0 (using 5 wt % NaOH) and maintained during the microbial treatment process. For accuracy, the experiment was repeated at 24-hour intervals for a total of 14 days.

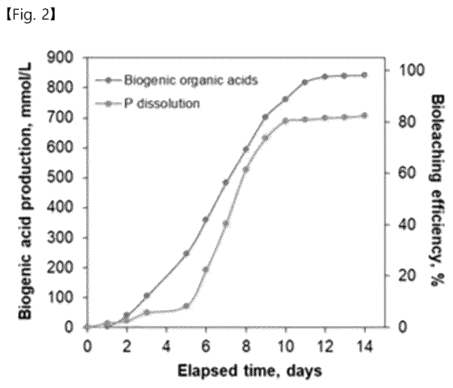

The concentration of metabolic acid produced and the bioleaching rate over time during the phosphorus leaching process for the monazite sample are shown in FIG. 2.

As can be seen from the drawings, production of metabolic acid was negligible until day 3, which indicates that no significant concentration of phosphorus was observed in the solution. However, 9 days after microbial treatment, about 840 mM of organic acid (metabolic acid), including 470 mM of oxalic acid, 300 mM of gluconic acid, and 70 mM of citric acid, was produced, which remained almost unchanged until day 14. In addition, the production of organic acid increased rapidly between day 3 and day 10, reaching >80%. Then, the phosphorus leaching efficiency did not change significantly until day 14 and reached 82.6%.

As such, even after long-term contact with the organic acid in the solution, there was no substantial change in the degree of phosphorus leaching in the sample, which indicates that the microorganisms passed the exponential growth phase and that phosphorus was not further required as an energy source to maintain microbial growth. In addition, it can be seen that 77% Na, 85% K, 81% Ca, 0.56% Y, 0.13% La, and 7.6% Fe were leached in addition to phosphorus during the leaching process.

### Example 2

Secondary bioleaching was performed on the residue left after phosphorus leaching in Example 1. At this time, a metabolic lixiviant was produced by the biochemical reaction of microorganisms using the spent medium, and the microorganisms used herein were a mixture of Alicyclobacilli and Sulfobacillus.

In addition, the microorganisms were cultured in a modified 9K medium and then supplemented with biological sulfur (collected from a sedimentation tank of a wastewater treatment plant) and sucrose as energy sources under aeration conditions of a temperature of 50° C. and a flow rate of 0.5 L/min. The L/S ratio was maintained at 2.5, and 1.0 L of the metabolic lixiviant produced at pH 1.5 was used for contact between 25 g of the iron-rich residue obtained in Example 1 and the spent medium. The bioleaching was performed in a 1.5 L bioreactor for 21 days under the conditions of a maintenance temperature of 50° C. and a stirring speed of 300 rpm. The leaching behavior of iron over time is shown in FIG. 3.

As can be seen from the drawing, 4 days and 6 days after biological leaching starts, iron leaching reached about 50% or more, and about 90%, respectively. Iron leaching reached 99.8% or more after 8 days. However, even after additional time elapsed, iron leaching exceeding about 4,848 mg/L was not observed (saturated state). It is considered that the saturated state resulted from the pH increase from an initial pH of 1.5 to a final pH of about 2.6. In addition to iron, most of Na, Ca, and K ions were leached. However, La and Y were leached to less than about 2%.

### Example 3

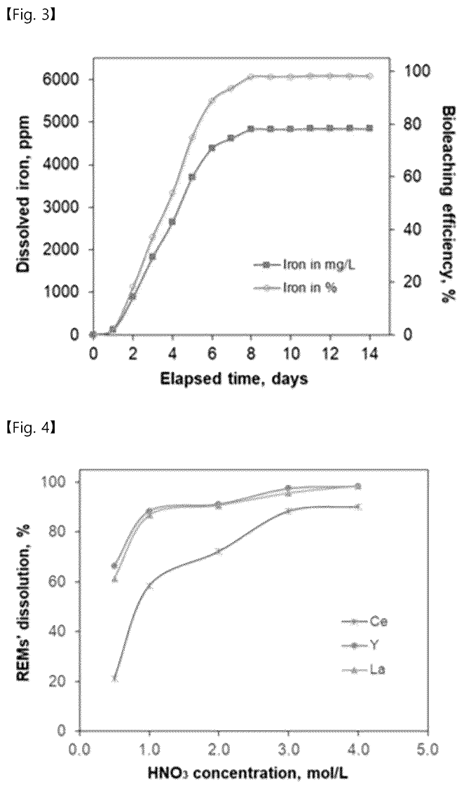

In order to leach rare earth metals (REM) in the iron-depleted residue obtained in Example 2, leaching was performed using a nitric acid solution in a sealed vessel, and ozone-nitration was further performed. As a result, the oxidation state of cerium was converted from Ce³⁺ to Ce⁴⁺. At this time, the gas was injected into a catalytic decomposer provided with a catalyst layer of CuO and MnO₂ for converting O₃ to O₂. 20 g of the iron-depleted residue was leached using nitric acid solutions of various concentrations (0.5 to 4.0 mol/L of HNO₃) under the conditions of ozone supply and a liquid-to-solid ratio of 10. In addition, the result was maintained for 4 hours under the conditions of an O₃ flow rate of 3.0 L/min and a temperature of 55° C. The leaching rates of three rare earth metals (Ce, La, and Y) depending on the concentration of nitric acid are shown in FIG. 4.

Referring to the drawing, when 0.5 mol/L of a HNO₃ solution was used, leaching of rare earth metals was very low. However, when 1.0 mol/L of HNO₃ is used, the leaching of rare earth metals greatly increased and, when 4.0 mol/L of HNO₃ was used, leaching of rare earth metals was maximized. In particular, when 4.0 mol/L of HNO₃ was used, more than 90% of Ce and more than 98% of Y and La were leached into the acid solution, and the concentrations of each rare earth metal in the leached solution were 1,046 mg/L of Ce⁴⁺, 688 mg/L of Y³⁺, and 384 mg/L of La³⁺.

### Example 4

The leachate obtained using 3.0 mol/L of HNO₃ in Example 3 contained 1,025 mg/L of Ce⁴⁺, 682 mg/L of Y³⁺, and 372 mg/L of La³⁺, and solvo-chemical separation and recovery of rare earth metals were performed on the leachate.

A pre-mixture of four organophosphorus compounds with various concentrations (0.05 to 0.25 mol/L) was pre-equilibrated to form a water immiscible extractant medium with different concentrations. In particular, the pre-mixture of the organophosphorus compound was prepared by mixing with 31 wt % of dioctyl-monohexyl P═O, 42 wt % of mono-octyl dihexyl P═O, 14 wt % of tri-hexyl P═O, and 8 wt % of trioctyl P═O as 5 vol % of a phase modifier in the presence of an aromatic solvent.

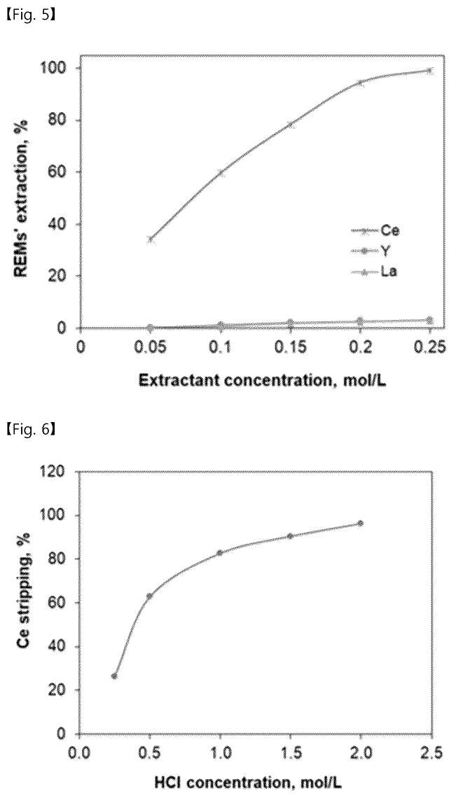

25 mL of rare earth metal leachate and 50 mL of a water-immiscible solvent mixture were brought in contact with a 100 mL glass separatory funnel. The aqueous and organic phases were separated from the solution which had been equilibrated at room temperature for an equilibration time of 5 minutes at which both the phases were settled (10 minutes). The bottom-settled aqueous stream was collected as a raffinate and the concentration of rare earth metals in the raffinate was analyzed after appropriate dilution. The extraction rates of three rare earth metals (Ce, La, and Y) in the leachate depending on the concentration of the organic extractant are shown in FIG. 5.

As can be seen from the drawing, quantitative extraction of cerium in the leachate was carried out (approximately 99%) through a single extraction step using 0.25 mol/L of an extractant in the organic phase. On the other hand, simultaneous quantitative extraction of lanthanum and yttrium was less than 3.5%. The +3 oxidation rare earth metals simultaneously extracted and nitric acid loaded in the organic phase were brought into with water and then separated by scrubbing.

### Example 5

The Ce(IV)-containing extract (organic solvent) obtained in Example 4 using an aqueous hydrochloric acid solution (2.0 M) was subjected to reduction stripping and the reducing agent added herein was H₂O₂ (0.1 mol/L).

50 mL of the cerium-containing extract and 25 mL of an aqueous hydrochloric acid solution were brought in contact with each other in a 100 mL separatory funnel. The aqueous and organic phases were separated from the solution equilibrated for 5 minutes at room temperature until both the phases were settled (10 minutes). The bottom-settled aqueous stream was collected as a raffinate and the cerium concentration was measured after appropriate dilution. The cerium stripping rate of the cerium-containing extract depending on the hydrochloric acid concentration (0.5 to 2.0 mol/L) during hydrochloric acid stripping using H₂O₂ (concentration: 0.1 mol/L) is shown in FIG. 6.

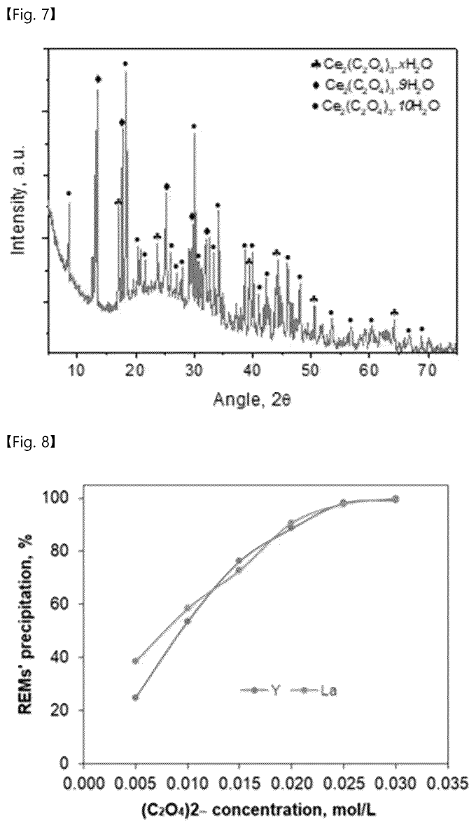

As can be seen from the drawing, the stripping of Ce (III) increased as the acid concentration increased, and a maximum of 96.2% of cerium was stripped with 2.0 mol/L HCl. In addition, when ammonium oxalate was added to the stripped solution, cerium oxalate was formed (see FIG. 7).

### Example 6

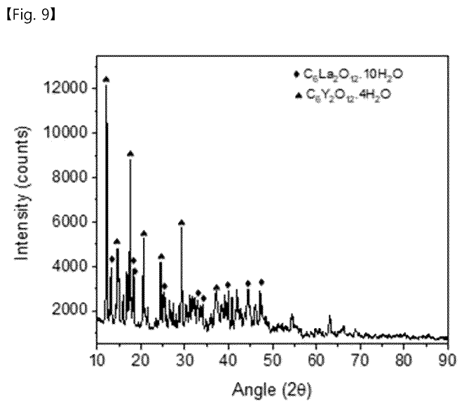

Ammonium oxalate was added as a precipitation salt to 100 mL of the raffinate generated after the cerium extraction in Example 4 to precipitate La³⁺ and Y³⁺. At this time, the precipitation conditions were set to a temperature of 90° C., a stirring speed of 150 rpm, and a contact time of 30 minutes. As a result, about 98% of the rare earth metal having an oxidation number of +3 was obtained using 0.025 mol/L of oxalate ions as a precipitate. In this regard, the precipitation rate of the rare earth metal depending on the concentration of oxalate ions during the process of forming the precipitate (oxalate precipitate) of rare earth metals (La, Y) other than cerium is shown in FIG. 8. In addition, the XRD pattern for the oxalate mixed precipitate of the rare earth metal (La, Y) is shown in FIG. 9.

As can be seen from the drawing, the precipitation amount of the oxalate salt of the rare earth mixture (La, Y) increased as the concentration of oxalate ions increased.

Simple modifications or changes of the present disclosure may be easily made by those skilled in the art and all such modifications or changes may fall within the scope of the present disclosure.

1. A method of recovering rare earth metals comprising:
a) culturing a phosphate solubilizing microorganism in the presence of a solid rare-earth metal-containing phosphate source, and leaching phosphorus in the solid rare-earth metal-containing phosphate source by metabolic acid discharged from the microorganism to form a phosphorus-containing leachate and a phosphorus-depleted residue,
wherein the rare-earth metal-containing phosphate source contains (i) cerium, (ii) at least one rare-earth metal other than cerium, and (iii) iron;
b) culturing a microorganism capable of oxidizing sulfur and iron in the presence of the phosphorus-depleted residue and leaching the iron in the phosphorus-depleted residue by a metabolic lixiviant discharged from the microorganism capable of oxidizing sulfur and iron to form an iron-depleted residue;
c) treating the iron-depleted residue with an acid to leach the rare earth metals and selectively oxidize cerium (III) among the rare earth metals and convert cerium (III) into cerium (IV);
d) extracting the cerium (IV) from the leachate obtained in step c) using an organic solvent to form a cerium-rich extract and a cerium-depleted raffinate; and
e) recovering cerium from the extract.

2. The method according to claim 1, wherein the rare earth metal-containing phosphate source contains 0.05 to 0.6 wt % of phosphorus (P), on an elemental basis.

3. The method according to claim 1, wherein the rare earth metal-containing phosphate source contains 0.5 to 4 wt % of the cerium, 0.1 to 3.5 wt % of the at least one rare earth metal other than cerium, and 5 to 30 wt % of the iron, on an elemental basis.

4. The method according to claim 3, wherein the rare earth metal-containing phosphate source contains up to 25 wt % of an oxide of other metal.

5. The method according to claim 3, wherein the rare earth metal-containing phosphate source is monazite.

6. The method according to claim 1, wherein the rare earth metal-containing phosphate source in step a) has a particle or pulverized material having a size ranging from 50 to 400 mesh.

7. The method according to claim 1, wherein the rare earth metal other than cerium comprises at least one of lanthanum (La) or yttrium (Y).

8. The method according to claim 3, wherein the other metal comprises at least one selected from the group consisting of silicon, titanium, aluminum, zirconium, sodium, potassium, calcium, manganese, and magnesium.

9. The method according to claim 1, wherein the phosphate solubilizing microorganism comprises at least one selected from the group consisting of the genus Aspergillus and the genus Penicillium, and
the microorganism capable of oxidizing sulfur and iron comprises at least one selected from the group consisting of the genus Alicyclobacillus and the genus Sulfobacillus.

10. The method according to claim 1, wherein, in step a), the metabolic acid comprises oxalic acid, and the concentration of oxalic acid in the metabolic acid is determined in a range of at least 200 mM.

11. The method according to claim 1, wherein, in step a), a part of the rare earth metal in the phosphate source is precipitated as an organic salt by the metabolic acid and is contained in the residue.

12. The method according to claim 1, wherein, the culture in step a) is performed in the presence of a microorganism initially cultured in a growth medium, wherein a liquid-solid ratio (L/S ratio) of the growth medium/phosphate source is adjusted in a range of 5 to 15.

13. The method according to claim 1, wherein step b) is performed using a medium supplemented with sulfur and nutrients,
wherein a liquid-to-solid ratio of the medium to the phosphorus-depleted residue is adjusted in a range of 2 to 5.

14. The method according to claim 1, wherein, in step c), the acid is nitric acid and ozone-nitration is involved.

15. The method according to claim 1, wherein, in step d), the organic solvent comprises an organophosphorus compound immiscible with water.

16. The method according to claim 15, wherein a concentration of the organophosphorus compound is in the range of 0.05 to 0.5 M,
wherein a volume ratio of an organic phase to an aqueous phase (O/A) is adjusted in a range of 5:1 to 1:5.

17. The method according to claim 1, wherein step e) comprises converting cerium (IV) into cerium (III) using hydrogen peroxide as a reducing agent and then stripping cerium (III) with an acid solution to obtain a cerium-containing acid solution.

18. The method according to claim 1, further comprising:
f) recovering rare earth metals other than cerium from the raffinate.

19. The method according to claim 17, further comprising precipitating, in the form of cerium oxalate, the cerium contained in the acid solution through the stripping, by use of oxalic acid ions to recover the cerium.

20. The method according to claim 18, wherein step f) further comprises precipitating, in the form of oxalate, rare earth metals other than cerium in the raffinate, by use of oxalic acid ions.
